(19) United States
(12) Patent Application Publication
(10) Pub. No.: US 2012/0065518 A1
(43) Pub. Date: Mar. 15, 2012

(54) SYSTEMS AND METHODS FOR MULTILAYER IMAGING AND RETINAL INJURY ANALYSIS

(75) Inventors: Rami Mangoubi, Newton, MA (US); Mukund Desai, Needham, MA (US); Kameran Lashkari, Boston, MA (US); Ahad Fazelat, Bedford, NH (US)

(73) Assignees: Schepens Eye Research Institute, Boston, MA (US); The Charles Stark Draper Laboratory, Inc., Cambridge, MA (US)

(21) Appl. No.: 13/233,520

(22) Filed: Sep. 15, 2011

Related U.S. Application Data
(60) Provisional application No. 61/403,380, filed on Sep. 15, 2010.

(51) Int. Cl. A61B 3/10; G06K 9/00

(52) U.S. Cl. 600/473; 382/128

(57) ABSTRACT

Systems and methods are provided for imaging an eye and identifying retinal or subretinal features that may be indicative of pathologies such as macular degeneration and traumatic brain injury. An infrared or near-infrared image of an eye is interdependently smoothed and segmented and attributes of the image are determined. The attributes are indicative of retinal or subretinal features.

### CROSS-REFERENCE TO RELATED APPLICATIONS

This application claims the benefit of U.S. Provisional Application No. 61/403,380, “Systems and Methods for Multilayer Imaging and Retinal Injury Analysis,” filed Sep. 15, 2010, and incorporated by reference herein in its entirety.

### STATEMENT REGARDING FEDERALLY SPONSORED RESEARCH OR DEVELOPMENT

Work described herein was funded, in whole or in part, by Grant No. RO1-EB006161-01A4 from the National Institutes of Health (NIH/NIBIB). The United States Government has certain rights in this invention.

### BACKGROUND OF THE INVENTION

Different imaging modalities capture different kinds of information about the structures being imaged. For example, infrared (IR) images of the retina and other multilayer structures contain information not available from visible light images because IR signals penetrate the multiple layers of the retina better than visible light. This is illustrated in FIG. 1, which depicts the depth to which light of various wavelengths (in nanometers) penetrates the retina. These wavelengths include visible wavelengths (denoted “RGB”) and IR wavelengths. In FIG. 1, reflections of visible light provide information from the retina, including the vitreo-retinal interface, while reflections of IR light provide information about the deeper tissue, including the photoreceptor-retinal pigment epithelium (RPE) interface, and the inner and outer choroidal areas. As such, IR images may contain information about sub-surface structures, such as subretinal structures, that can be used for early and improved diagnosis of, for example, subretinal pathologies and injury-related changes in the eye. Such sub-surface structures are sometimes clouded by the opacities of the vitreous humor, hemorrhage, and other media opacities such as cataracts (which obstruct the path of visible light but allow penetration of IR light). FIG. 2 compares a visible image (left) and an IR image (right) of a dilated eye. While the retinal surface vasculatures can be seen in the visible light image, the IR image can suggest the presence of lesions 4 and 6 not seen in the visible image.

However, because IR light can penetrate and reflect from multiple layers of a structure, IR images are hard to read with the human eye. IR images present two principal challenges. First, IR images are often of lower spatial resolution than visible light images and consequently are more muddied and less sharp. Second, the intensity at each pixel or pixel region consists of information from multiple layers of the structure (e.g., the retinal pigment epithelium and choroidal areas of the retina), reducing contrast for subretinal features. A clinician using existing imaging and analysis systems cannot distinguish the information arising from each different layer of the structure, and thus many of the advantages of multilayer imaging have not been realized.

The difficulty of extracting information from IR images has impeded the adoption of IR imaging, particularly in retinal fundus imaging. In current practice, a clinician interested in the surface and deeper tissue of a patient's eye will dilate the patient's eye prior to visible light imaging, and/or contrast the patient's blood, in order to detect and locate retinal/choroidal structures. However, these are invasive and uncomfortable procedures for the patient. Existing IR imaging systems, such as scanning laser ophthalmoscopes, are expensive, fragile, difficult to transport and require significant operator training.

### SUMMARY

Described herein are systems, methods and non-transitory computer readable media for multilayer imaging and retinal injury analysis. These methods are preferably implemented by a computer or other appropriately configured processing device.

In a first aspect, a computer receives a first image of an eye of a subject, the first image including at least one infrared image or near-infrared image. The computer interdependently smoothes and segments the first image. Segmenting the first image may include identifying edge details within the first image. The segmenting and smoothing may include determining an edge field strength at a plurality of locations in the image, and the computer may determine the attribute based on the edge field strength. In some such implementations, the edge field strength is based on a matrix edge field.

The computer determines a value of an attribute at a plurality of locations within the smoothed, segmented first image. The attribute is indicative of at least one retinal or subretinal feature in the first image. In some implementations, the computer identifies the at least one feature based at least in part on the first attribute image. The computer may identify a boundary of the at least one feature based at least in part on the first attribute image. The at least one feature may include a lesion, and the computer may provide quantitative information about the lesion. The feature may include a zone 3 injury, such as a choroidal rupture, a macular hole, or a retinal detachment. The feature may be indicative of a traumatic brain injury. The feature may be indicative of at least one of age-related macular degeneration, juvenile macular degeneration, retinal degeneration, retinal pigment epithelium degeneration, toxic maculopathy, glaucoma, a retinal pathology and a macular pathology.

The computer generates a first attribute image based at least in part on the determined values of the attribute, and provides the first attribute image. In some implementations, the computer provides the first attribute image to a display device, and may subsequently receive a triage category for the subject from a clinician. In some implementations, the computer provides a sparse representation of the first attribute image. To provide the sparse representation, the computer may perform a compressive sensing operation. The computer may also store a plurality of features on a storage device, each feature represented by a sparse representation, and may compare the identified at least one feature to the stored plurality of features.

In some implementations, the computer also receives a second image of the eye, the second image generated using a different imaging modality than used to generate the first image of the eye. The imaging modality used to generate the second image of the eye may be visible light imaging, for example. In such an implementation, the computer combines information from the first image of the eye with information from the second image of the eye. The computer may also display information from the first image of the eye with information from the second image of the eye. In some implementations, the computer combines information from the first image of the eye with information from a stored information source.

In some implementations, the computer determines a textural property of a portion of the first image of the eye based at least in part on the first attribute image, and also compares the first image of the eye to a second image of a second eye by comparing the determined textural property of the portion of the first image of the eye to a textural property of a corresponding portion of the second image of the second eye. The first image and the second image may represent a same eye, different eyes of a same subject, or eyes of different subjects. For example, the first image and the second image may represent a same eye at two different points in time. The computer may be included in a physiological monitoring or diagnostic system, such as a disease progression tracking system, a treatment efficacy evaluation system, or a blood diffusion tracking system. In some implementations, the textural properties of the respective portions of the first and second images of the eye are represented by coefficients of a wavelet decomposition, and the computer compares the first image of the eye to the second image of the eye comprises comparing the respective coefficients for a statistically significant difference. In some implementations, the textural properties of the respective portions of the first and second images are represented by respective first and second edge intensity distributions, and the computer compares the first image of the eye to the second image of the eye comprises comparing at least one statistic of the first and second edge intensity distributions.

### BRIEF SUMMARY OF THE DRAWINGS

The patent or application file contains at least one drawing executed in color. Copies of this patent or patent application publication with color drawings will be provided to the Office upon request and payment of the necessary fee. The above and other features of the present invention, its nature and various advantages will be more apparent upon consideration of the following detailed description, taken in conjunction with the accompanying drawings in which:

FIG. 1 is a diagram of the physics of visible and IR imaging of the eye;

FIG. 2 is a comparison between a visible image (left) and an IR image (right) of a dilated eye;

FIG. 3 is a schematic of an automated decision support system according to an illustrative embodiment;

FIG. 4 is a diagram of an IR indirect fundus camera that may be included in the automated decision support system of FIG. 3 according to an illustrative embodiment;

FIG. 5 is a flow chart of a process for imaging an eye with the automated decision support system of FIG. 3 according to an illustrative embodiment;

FIGS. 6-9 depict examples of attribute images generated by the process of FIG. 5 according to illustrative embodiments;

FIG. 10A depicts visible and IR retinal images before and after processing with the process of FIG. 5 in accordance with an illustrative embodiment;

FIG. 10B depicts information from multiple imaging modalities overlaid in a combined image in accordance with an illustrative embodiment;

FIG. 11 is a flow chart of a process for comparing first and second fundus images with the automated decision support system of FIG. 3 according to an illustrative embodiment;

FIGS. 12 and 13 depict examples of time-based image analyses resulting from the process of FIG. 11 according to an illustrative embodiment;

FIG. 14 demonstrates the improvements achievable by the automated decision support system of FIG. 3 in the detection of fine features in fundus images in the presence of noise;

FIG. 15 is a diagram of an illustrative process for generating smoothed data, segments, and attribute estimates from one or more images according to an illustrative embodiment;

FIGS. 16A-16C depict three different approaches to neighborhood adaptation for smoothing and segmenting according to an illustrative embodiment; and

FIG. 17 illustrates a process for generating smoothed data, segments and attribute estimates by minimizing an energy function according to an illustrative embodiment;

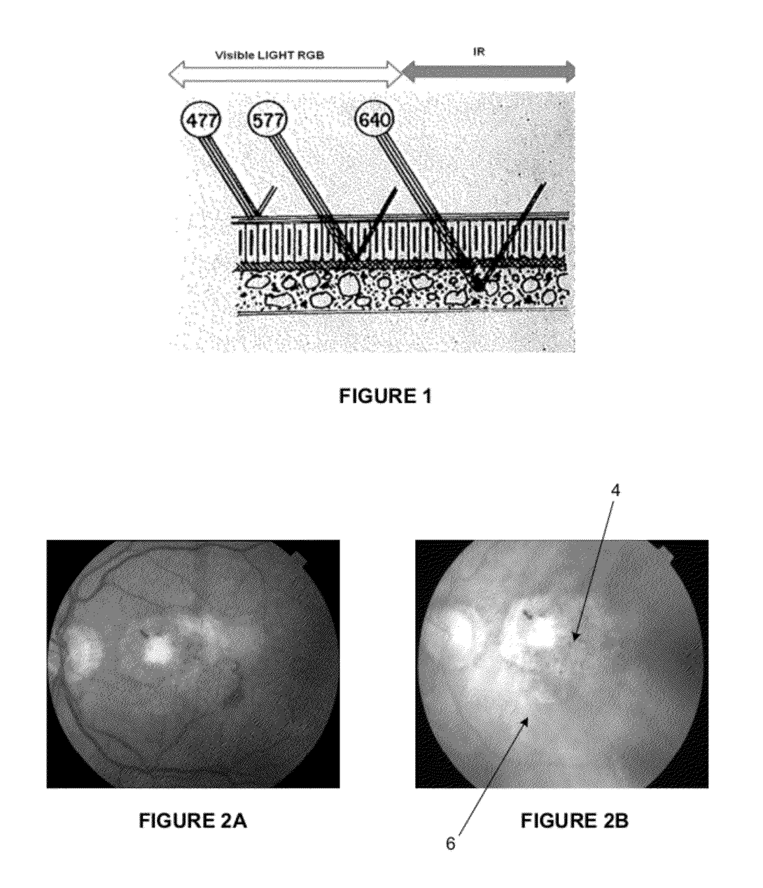

### DETAILED DESCRIPTION

To provide an overall understanding of the invention, certain illustrative embodiments will now be described, including systems and methods for computing and scoring the complexity of a vehicle trip using geo-spatial information. However, it will be understood by one of ordinary skill in the art that the systems and methods described herein may be adapted and modified as is appropriate for the application being addressed and that the systems and methods described herein may be employed in other suitable applications, and that such other additions and modifications will not depart from the scope thereof.

### A. Automated Decision Support Systems

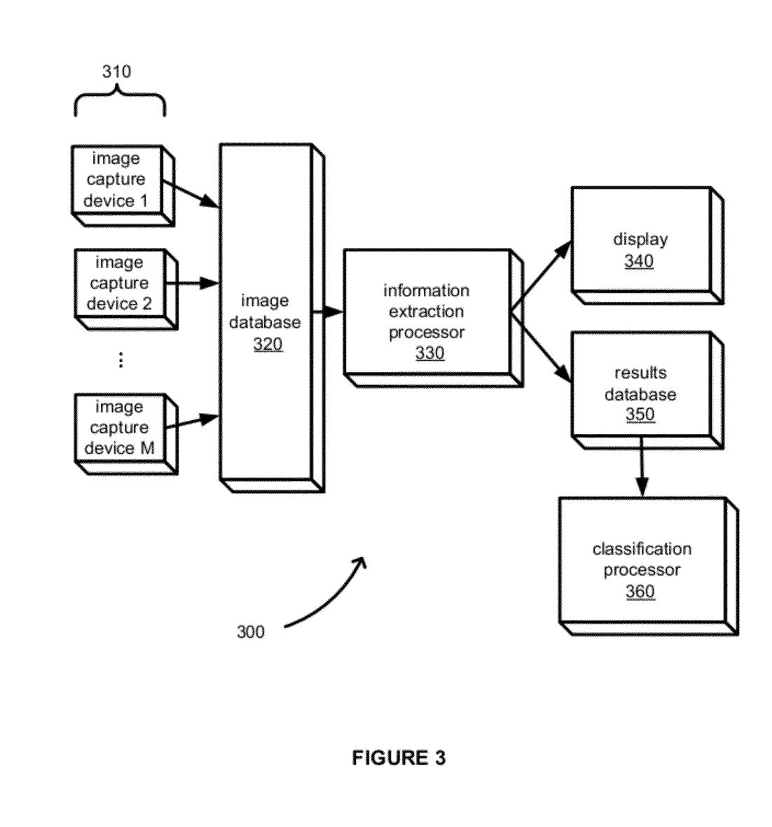

FIG. 3 is a schematic diagram of an automated decision support system, according to an illustrative implementation. The system 300 includes a number of functional components, including image capturing devices 310, image database 320, information extraction processor 330, display 340, results database 350 and classification processor 360. The functional components of the system 300 may be implemented in any combination with any of the other functional components (as well as with any additional components described herein). Each component can be implemented in any suitable combination of hardware (e.g., microprocessors, DSP chips, solid-state memory, hard drives, optical drives and removable media) and software (i.e., computer-readable media) configured to perform the functions described herein. The functions of each functional component may also be distributed across a number of separate hardware components, or implemented by any number of software modules. Each of the functional components of the automated decision support system 300 of FIG. 3 is now discussed.

The system 300 includes at least one image capture device 310 for capturing images of a scene. Exemplary image capture devices 310 include visible light cameras and video recorders, images captured by various scanning laser ophthalmoscopes with or without dye in all wavelengths, optical coherence tomography, PET, SPECT, MRI, X-ray, CT scanners and other medical imaging apparatus; bright field, phase contrast, atomic force and scanning electron microscopes; satellite radar; thermographic cameras; seismographs; and sonar and electromagnetic wave detectors. Each of the image capturing devices 310 may produce analog or digital images. The image captured by a single image capturing device 310 may be scalar-, vector- or matrix-valued and may vary as a function of time. An imaged scene can include any physical object, collection of physical objects or physical phenomena of interest for which measurements of at least one property can be obtained by an image capturing device. For example, the embryonic environment of a fetus is a scene that can be measured with an ultrasound image capture device. In another example, the position and movement of atmospheric moisture is a scene that can be measured with a satellite radar image capture device.

An image database 320 is used to store the images captured by the image capturing devices 310 as a set of image data. Image database 320 may comprise an appropriate combination of magnetic, optical and/or semiconductor memory, and may include, for example, RAM, ROM, flash drive, an optical disc such as a compact disc and/or a hard disk or drive. One skilled in the art will recognize a number of suitable implementations for image database 320 within system 300, with exemplary implementations including a database server designed to communicate with processor 330, a local storage unit or removable computer-readable media.

Information extraction processor 330 and database 320 may be embedded within the same physical unit or housed as separate devices connected to each other by a communication medium, such as a USB port, serial port cable, a coaxial cable, a Ethernet type cable, a telephone line, a radio frequency transceiver or other similar wireless or wired medium or combination of the foregoing. Information extraction processor 330 queries database 320 to obtain a non-empty subset of the set of image data. Information extraction processor 330 also performs the information extraction processes described below. Exemplary implementations of information extraction processor 330 include the software-programmable processors found in general purpose computers, as well as specialized processor units that can be embedded within a larger apparatus. Information extraction processor 330 performs the method described herein by executing instructions stored on a computer-readable medium; one of ordinary skill in the art will recognize that such media include, without limitation, solid-state, magnetic, holographic, magneto-optical and optical memory units. Additional implementations of information extraction processor 330 and the remaining elements of FIG. 3 are discussed below.

At the completion of the information extraction method, or concurrently with the method, information extraction processor 330 outputs a collection of processed data. Display 340 presents the processed data visually to a user; exemplary implementations include a computer monitor or other electronic screen, a physical print-out produced by an electronic printer in communication with information extraction processor 330, or a three-dimensional projection or model. Results database 350 is a data storage device in which the processed data is stored for further analysis. Exemplary implementations include the architectures and devices described for image database 320, as well as others known to those skilled in the art. Classification processor 360 is a data processing device that may optionally extract the processed data from database 350 in order to classify the processed data, i.e. identify the meaning and content of elements in the imaged scene, and may be embodied by the architectures and devices described for information extraction processor 330.

Although the system components 310-360 are depicted in FIG. 3 as separate units, one skilled in the art would immediately recognize that two or more of these units could be practically combined into an integrated apparatus that performs the same overall function. For example, a single physical camera may have both visible and IR imaging capabilities, and thus represent two image capture devices. A single image processing device may also contain a database 320 for the image data, which can be directly transmitted to processor 330. Similarly, the database 320 and the processor 330 could be configured within a single general purpose computer, as could the processors 330 and 160. Many combinations of the system components within hardware are possible and still remain within the scope of the claimed system. The system components 310-360 can be coupled using communication protocols over physical connections or can be coupled using wireless communication protocols. In one exemplary implementation, the image data is transmitted from remote image capture devices wirelessly or via an electronic network connection to a data processing facility, where it is stored and processed. In another exemplary implementation, the system of FIG. 3 is deployed within a vehicle or fleet of vehicles which is capable of using the processed data to make decisions regarding the vehicle or fleet's behavior.

Returning to FIG. 3, one skilled in the art will recognize that many different implementations of the system components 310-360 are possible, as are many different settings for the system as a whole. In one implementation, the system of FIG. 3 resides in a laboratory or medical research setting and is used to improve patient diagnosis using image data from, for example, perfusion imaging, fMRI, multi-spectral or hyper-spectral imaging, bright field microscopy or phase contrast microscopy. In another implementation, the system of FIG. 3 resides in a monitoring station and is used to assess conditions in a particular geographical area by combining data from at least one imaging device. These devices may include satellite radar, aerial photography or thermography, seismographs, sonar or electromagnetic wave detectors. In another implementation, the system of FIG. 3 can be configured for any general purpose computer to meet the hardware requirements and extract information from image data arising from a user's particular application.

### B. Eye Imaging and Feature Extraction

As described above, whole-field, IR fundus illumination may reveal significantly more detail about retinal, subretinal and choroidal anatomy than visible light imaging, but employing a long-wavelength light source and illuminating the entire fundus may yield suboptimal contrast and resolution. IR fundus images are a composite of light that is reflected or back-scattered and light that is absorbed by ocular pigment, choroid and hemoglobin. However, when the entire fundus is illuminated, the contrast of any object-of-interest is degraded by light that is scattered by superficial, deep and lateral structures. Light is therefore multiply-scattered and contrast is degraded making edge detection extremely difficult. Conventional IR image analysis systems extract only a small fraction of the clinically-relevant information that is embedded in IR fundus images of the eye. There is a need for systems and techniques for parsing and analyzing the multilayer information of IR and other multilayer imaging modalities. Disclosed herein are systems and techniques for:
extracting information from and enhancing IR images of the retina or other multilayer structure;
analyzing such images in time and space to detect and locate anomalies (such as retina lesions); and
using such images, assisting in the diagnosis of traumatic injuries, age-related macular degeneration, other types of RPE, photoreceptor and macular degeneration, toxic maculopathy, glaucoma, trauma, or other causes of damage to a single or multilayer structure.

This disclosure presents a number of systems and techniques that can be applied in imaging applications in which information is to be captured from different layers of a multilayer structure. Some of the information extraction and analysis techniques described herein use one or more original images of a structure to generate one or more output images that provide information about user-specified attributes of the structure (for example, high frequency details of the structure, presented at different spatial scales and intensities). As described above, the multilayer information in the output images is not visible in the original images. The systems and techniques described herein enable fine, high-frequency details to be extracted from IR and near-IR images. The systems and techniques described herein are capable of extracting information from multiple depths in a structure by analyzing images taken captured using multiple different wavelengths of light or a single image captured using multiple different wavelengths of light (followed by post-processing to extract the features distinguished by the multiple wavelengths), as well as other combinations of different imaging modalities.

In some implementations of the systems and techniques described herein, the output images described above are further processed to identify particular features; for example, features indicative of lesions in retinal or subretinal layers. The improvements in retinal and subretinal feature identification achieved by the systems and methods described herein enable the effective triage and treatment of eye injuries. Effective triage may be especially important in military settings. For example, a study of soldiers evacuated from Operation Iraqi Freedom and Operation Enduring Freedom with eye injuries was performed from March 2003 through December 2004 (Aslam and Griffiths, “Eye casualties during Operation Telic,” J. R. Army Med. Corps., March 2005, pp. 34-36). Data came from the Military Office of the Surgeon General (OTSG) Patient Tracking Database. A total of 368 patients (451 eyes) were evacuated for eye-related problems: 15.8% (258 of 1,635 patients, 309 eyes) of all medical evacuations were a result of battle eye injuries (BI), 17.3% (283 of 1,635 patients, 337 eyes) were a result of eye injuries (BI and non battle injuries [NBI] combined), and 22.5% (368 of 1,635 patients, 451 eyes) of all evacuations were at least partly due to eye-related complaints. Even worse, many incipient and subtle lesions and injuries are not identified due to lack of facilities. If undiagnosed and untreated for even a few hours, the likelihood of permanent retina damage increases. The ability to analyze fundus images on site in real time may enable effective triage and selection for transport to regions where eye specialists are available, thereby improving the efficiency of current ocular telemedicine systems and the standard of care for soldiers.

In some implementations, lesions indicative of traumatic brain injuries (TBI) are automatically identified. Existing TBI identification systems and techniques require expensive, difficult-to-field modalities, such as MR imaging (requires extensive testing facilities and trained clinicians), scanning laser ophthalmoscope (SLO) imaging, and ICG angiography (typically used for choroidal vasculature, but often produces highly noisy results). Subjective assessments from patient examination are also used, but tend to be unreliable. The improved techniques described herein for identifying trauma-related eye lesions allow the early discovery of the retina or choroid which may correlate strongly with concomitant TBI.

Also described herein is an automated decision support system that includes or communicates with a multilayer imaging system (e.g., an IR imaging system). This system uses the techniques described herein to image, analyze and triage a patient's condition, providing faster and improved diagnosis of TBI and other injuries. In certain implementations, this automated decision support system is embedded, along with an imaging system, in a portable device that can be used in battlefield or accident scenarios.

For clarity of description, this disclosure uses the example application of IR retinal imaging to illustrate some of the particular features and advantages of these systems and techniques. The term “IR” is used herein to refer to infrared and near-infrared wavelengths. However, these systems and techniques are not limited to retinal imaging applications nor IR wavelengths, and are readily applied to any suitable medical or non-medical multilayer imaging application at any wavelengths. Additionally, the systems and techniques described herein are applicable to other imaging modalities, such as optical coherence tomography (OCT), laser, scanning laser ophthalmoscope (SLO) and ICG angiography, among others.

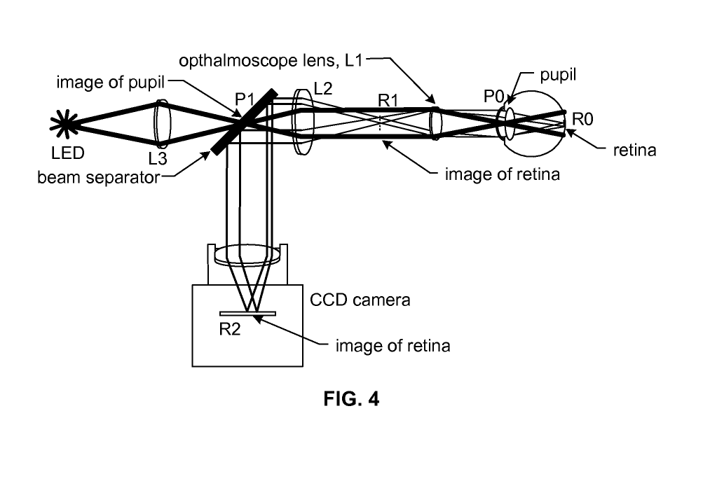

Systems and methods for multilayer feature extraction of eye images are now discussed. In some applications, the components of the automated diagnostic support system 300 (FIG. 3) may be selected to be suitable for multilayer feature imaging, and may include any imaging system capable of capturing one or more images from which information about different layers of a multilayer structure may be extracted. For example, the image capture device 310 of the system 300 may be an analog or digital IR fundus camera which is capable of imaging the interior surface of the eye (e.g., the retina, optic disc, macula and posterior pole) with IR wavelengths. The image capture device 310 may be used with light of one or more wavelengths in the IR range, or near the IR range, and may include optical filters for passing or blocking different wavelengths of interest. For example, FIG. 4 is a diagram of an IR indirect fundus camera that can be used with the systems and techniques described herein, but any camera capable of imaging the structure of interest with the appropriate imaging modality may be used. In some applications, preferred cameras produce digital IR fundus images that can be used with the information extraction and analysis techniques described herein. For example, a Topcon TRC 501X tri-functional fundus camera may be used, and with a 20-, 30-, or 50-degree field to acquire color, red-free and near-IR images of a subject's eye. These images may be captured without the need for any intravenous dye, such as fluorescein isothyacyante or indocyanine green. Fundus cameras may be used in mydriatic diagnoses, in which a patient's eye is dilated with eye drops prior to imaging, or in non-mydriatic diagnoses. Additional examples of image capture devices are described by Harooni and Lashkari in U.S. Pat. No. 5,841,509, U.S. Pat. No. 6,089,716, and U.S. Pat. No. 6,350,031, each of which is incorporated by reference in its entirety herein.

As described above with reference to FIG. 3, the image captured by the image capturing device 310 may be scalar-, vector- or matrix-valued and may vary as a function of time. Eye images can be stored in an image database 320 for real-time or later processing, analysis and review by information extraction processor 330, display 340, and results database 350 (FIG. 3). These components can take the form of any of the implementations described herein. The information extraction processor 330 includes one or more processors and memory devices, and is configured to perform any one of more of the feature or multilayer extraction techniques described in detail in Sections C-E, below.

The automated decision support system 300 may also be configured to send and/or receive information between the automated decision support system 300 and a remote location (or a local, separate device). The automated decision support system 300 may use any wired or wireless communications protocol to send images and information. In certain implementations, the automated decision support system 300 manages communication between a portable automated decision support system and a base station or command center and enables communication between remotely-located clinicians in a battlefield setting. In certain implementations, the automated decision support system 300 is configured to retrieve previously-stored information about a patient under study, which can inform a clinician of a change in a patient's condition or provide additional factors for the clinician or the automated decision support system 300 to consider. The display 340 provides a way of informing a patient or care provider of the results of the imaging and analysis techniques described herein, and may include any device capable of communicating such information, including a visual monitor, an audio output, an electronic message to one or more receiving devices, or any combination of such displays.

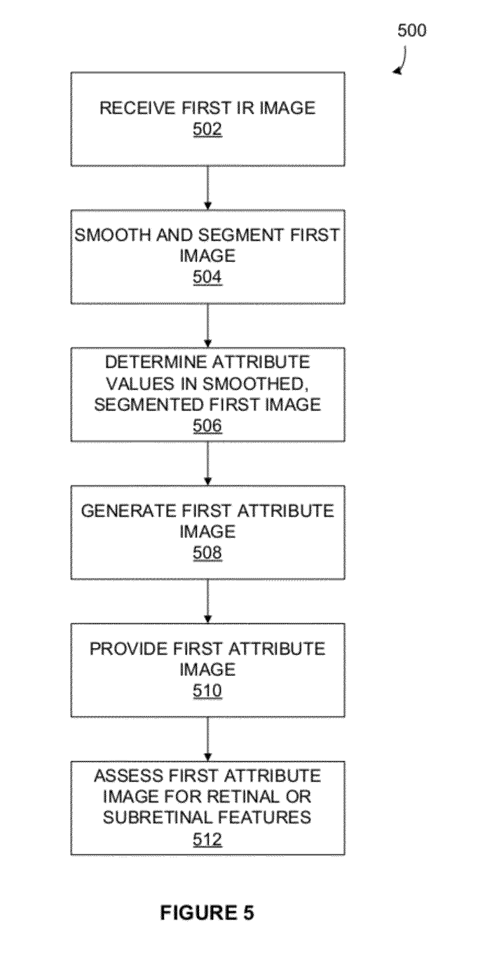

FIG. 5 is a flow chart of a process 500 that may be executed by the automated decision support system 300 (FIG. 3). The process of FIG. 5 is illustrated (and will be described) as applied to IR imaging applications, but any imaging modality may be used. At the step 502, the system 300 (FIG. 3) receives a first image of an eye of a subject. The image received at the step 502 is generated by an image capture device using electromagnetic radiation with IR or near-IR wavelengths. As indicated above, the range of IR and near-IR wavelengths is referred to collectively as “IR” herein, and the first image may be referred to as an “IR” image. The first image is captured by a fundus camera or any other imaging device, and may be illuminated at any IR or near-IR wavelength.

At the step 504, the system 300 performs a smoothing and segmenting technique, which may take the form of any of the smoothing, segmenting and attribute estimation techniques described herein (including those described in Sections C-E, below). As described below, the smoothing and segmenting operations in these techniques may be interdependent and performed substantially simultaneously (e.g., in frequent alternating iterations). In some implementations, the segmenting and smoothing technique performed at step 504 involves determining a smoothed image and/or edge details (such as an edge field strength) at a plurality of locations in the image.

The edge field may be a scalar-valued edge field (e.g., as illustrated in Eq. 5), a vector-valued edge field (e.g., as illustrated in Eq. 6), a matrix-valued edge field (e.g., as illustrated in Eq. 7), or any combination thereof. In some implementations, the smoothing and segmenting technique performed at the step 504 includes adaptively adjusting at least one of a shape and orientation defining a neighborhood associated with a plurality of locations in the image. The system 300 may perform the segmenting and smoothing technique to reduce the value of an energy function associated with an error metric, as discussed in detail in Sections C-E, below. A detailed description of several particular implementations of step 504 follows.

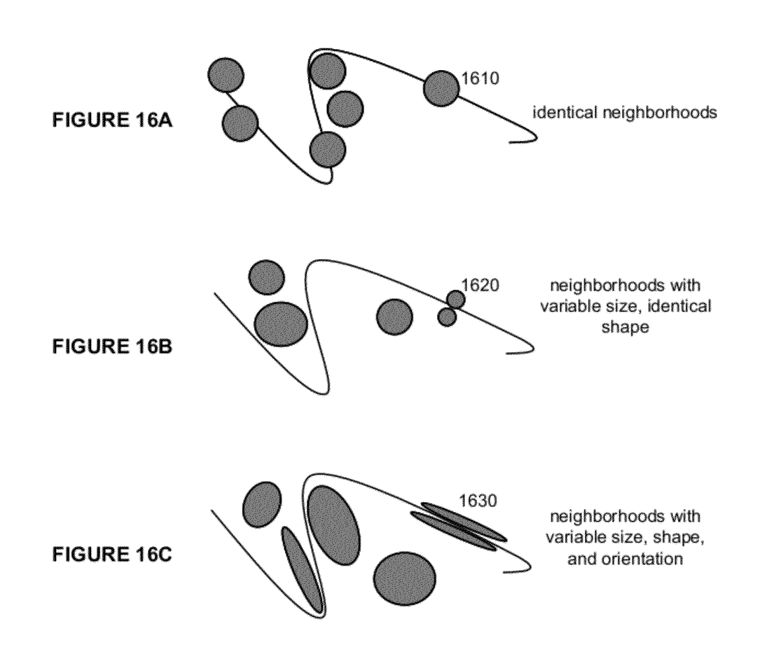

In some implementations, each location in the image is associated with a elliptical neighborhood over which the image is smoothed and an edge field estimated, and the size, shape and orientation of the neighborhood vary from location to location. The neighborhoods of locations identified as edges (e.g., blood vessel edges) are adaptively reduced in size to limit the “blurring” of the edge by smoothing across the edge (e.g., as illustrated in FIG. 16C). This formulation can be expressed as:

$${min}_{u , V} {\int }_{R} [ α {{u}_{X}^{T} ( 1 - V )}^{2} {u}_{X} + {β ( u - g )}^{2} + \frac{ρ}{2} F ( {V}_{X} ) + \frac{G ( V )}{2 ρ} ] d X \qquad ( 1 )$$

wherein g is the retinal image data output by the image capture device 310, u is the smoothed data, V is a 2×2 symmetric edge matrix field, X is the image over which the smoothing and segmenting takes place, and α, β, ρ are adjustable parameters. As described in detail in Sections C-E, the first term can be interpreted as a smoothness fidelity term that penalizes the gradient of u by I-V, so that smoothing occurs primarily based on pixels situated in the neighborhood. The second term is a data fidelity term penalizing deviations of the smoothed image data from the input image data. The scalar term G(V) penalizes edge strength, while F(V_{X}) balances a preference for smooth edges with high-frequency features that may be present in the first image.

While any numerical technique may be used to solve Eq. 1 for any particular image and parameter values, one approach includes the use of the Euler-Lagrange equations that form the basis of the solution. For Eq. 1, the Euler-Lagrange equations are:

$$\begin{matrix} ∇ ( {( I - V )}^{T} ( I - V ) {u}_{X} ) - \frac{β}{α} ( u - g ) = 0 & ( 2 ) \\ {\sum }_{i = 1}^{2} \frac{\partial }{\partial {x}_{i}} ( \frac{\partial F ( {V}_{X} )}{\partial {V}_{{X}_{i}}} ) - \frac{α}{β} [ ( I - V ) {u}_{X} {u}_{X}^{T} + {u}_{X} {u}_{X}^{T} ( I - V ) ] + \frac{1}{{ρ}^{2}} \frac{\partial G ( V )}{\partial V} = 0 & ( 3 )\end{matrix}$$

Additional or alternate smoothing and segmenting approaches may also be applied at the step 504, such as standard spatial filtering and denoising techniques.

At the step 506, the system 300 determines a value of an attribute at a plurality of locations within the first image. The attribute is indicative of at least one retinal or subretinal feature in the first image.

At the step 508, the system 300 generates a first attribute image based on the attribute determination of the step 506. Specifically, for each attribute of interest, an image is generated indicating the value of that attribute for each pixel in the original image. This image may serve as the attribute image generated at the step 508, or may be further processed to generate the attribute image. If N attributes are determined at the step 506, then N attribute images may be generated at the step 506. In certain implementations, less or more than N attribute images are generated, and each attribute image may include a combination of information from two or more attribute images.

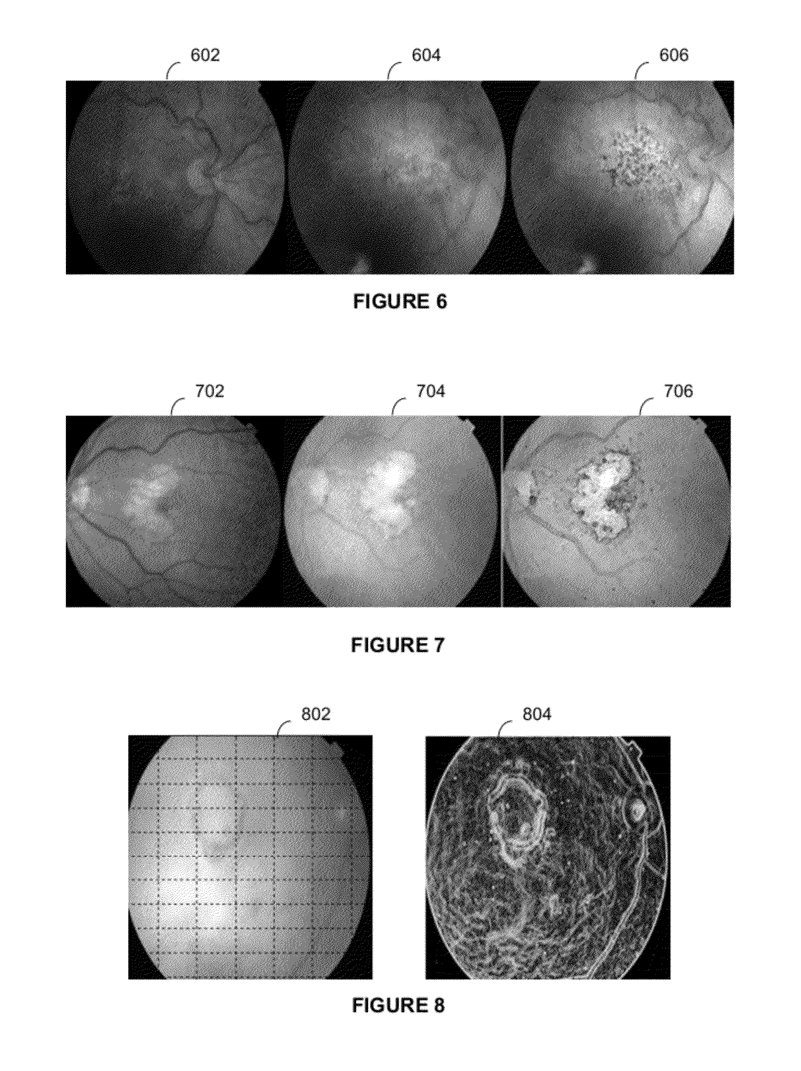

Several examples of attribute images generated at the step 506 (FIG. 5) are now discussed with reference to FIGS. 6-9. In these examples, the attribute of interest is the presence of edges (which may capture, e.g., high frequency spatial details), with the intensity of the image at a pixel related to the value of the edge-field at that pixel. A first example of an attribute image generated at the step 506 (FIG. 5) is illustrated in FIG. 6, in which the leftmost image 602 is a visible light image of a retina, the middle image 604 is a “raw” IR image of the retina before the processing of the step 504, and the rightmost image 606 is an attribute image generated from the raw IR image at the step 506. The color fundus image 602 and the raw IR image 604 indicate the presence of macular drusen and pigment clumping associated with intermediate dry age-related macular degeneration (AMD). The scatter of increased reflectance in the macular region of the IR image 604 indicates chorioretinal atrophy. In the attribute image 606, these features are more clearly defined and their extent highlighted: the dark, stippled area in the perifoveal region represents pigment clumping and variable bright areas in the macular region represent increased reflectance likely due to chorioretinal atrophy. These features can thus be analyzed by a clinician or the automated decision support system 300, as discussed below.

A second example of an attribute image generated at the step 506 (FIG. 5) is illustrated in FIG. 7, in which the leftmost image 702 is a visible light image of a retina, the middle image 704 is a raw IR image of the retina, and the rightmost image 706 is an attribute image generated from the raw IR image at the step 506. The color fundus image 702 and the raw IR image 704 indicate the presence of geographic atrophy, scattered macular drusen and pigment clumping associated with advanced dry AMD. Increased reflectance in the macular region of the IR image 1104 indicates chorioretinal atrophy. In the attribute image 706, the geographic area of increased reflectance indicates chorioretinal atrophy with clearly delineated geographic borders.

A third example of an attribute image generated at the step 506 (FIG. 5) is illustrated in FIG. 8, in which the left image 802 is a raw IR image of the retina and the right image 804 is an attribute image generated from the raw IR image at the step 506. The attribute image 1106 enhances the choroidal neovascular membrane associated with a wet form of AMD, identifying its contours and a leak in the membrane itself.

Returning to the information extraction and analysis process depicted in FIG. 5, once the first attribute image is generated at the step 508, the attribute image is provided at the step 510. In some implementations, the system 300 further processes the first attribute image at the step 510 to extract features of the structure being imaged and/or model the condition of the structure. In some implementations, the system 300 is configured to identify at least one retinal or subretinal feature based at least in part on the first attribute image generated at the step 1008. In certain implementations, the step 510 includes executing lesion detection techniques that detect and locate lesions or other anomalies in the attribute images. In retinal imaging applications, the step 510 preferably includes identifying lesions in retinal or subretinal layers. For example, when the first attribute image represents the edge-field strength of the smoothed and segmented first image, the system 300 may apply a boundary-identification technique to isolate retinal or subretinal features of interest (e.g., lesions) from the surrounding areas. Once the boundary of the feature has been identified, the system 300 may determine quantitative information about the lesion (e.g., spatial properties of the feature, such as its area and perimeter). These quantities may be used by the system 300 to diagnose and triage the subject, or may be displayed for a clinician.

At the step 512, the features extracted and models built at the step 510 are used by the automated decision support system 300 (FIG. 3) to provide some assessment of the imaged structure. Many different kinds of assessments may be made at the step 512 based on the first attribute image generated at the step 508. In some implementations of the step 512, the system 300 analyzes the first attribute image and extracts one or more baseline features indicative of the general health of a patient's eye (e.g., the retina and choroid). These baseline features are used as a point of comparison for future clinical follow-up. In practice, the systems and techniques described herein have been used on clinical data to non-invasively extract detailed information about many pathological conditions that were previously difficult to identify, including commotio retinae, choroidal rupture, choroidal effusion, choroidal hemorrhage, various forms of choroidal neovascular membranes including classic, occult and mixed forms, subretinal heme, subretinal fluid, retinal pigment proliferation and migration, pigment epithelial detachment, pigment epithelial tears, subretinal drusen, retinal angiomatous proliferation, and lesions associated with idiopathic polyploidal vasculopathy, among other features. Areas with enhanced, diminished, or absent retinal or fundus fluorescence can be measured and quantified in various wavelengths. In some implementations, the system 300 is configured to identify feature indicative of one or more of age-related macular degeneration, retinal degeneration, retinal pigment epithelium degeneration, toxic maculopathy, glaucoma, a retinal pathology and a macular pathology.

For example, the system 300 may be configured to detect a lesion at the step 512. In some implementations, a lesion is characterized by its grade or severity (particularly those lesions due to macular degeneration and traumatic retinal injuries) according to clinician-specified or learned criteria. In some implementations of the step 512, the system 300 indicates that a traumatic brain injury feature has been observed. As described above, traumatic brain injuries (TBIs) are detected by identifying certain retinal and subretinal features that characterize a zone 3 injury (such as a choroidal rupture, a macular hole, or a retinal detachment). In some implementations, when the system 300 identifies any features related to a potential TBI, the system 300 provides the attribute image to a clinician display and, optionally, further provide a message indicating that a potential TBI has been detected. In some implementations, the system 300 indicates the location of the TBI-related feature on the attribute image. In response, a clinician may input a triage category for the subject into the system 300 to route the subject into appropriate channels for additional medical care or follow-up.

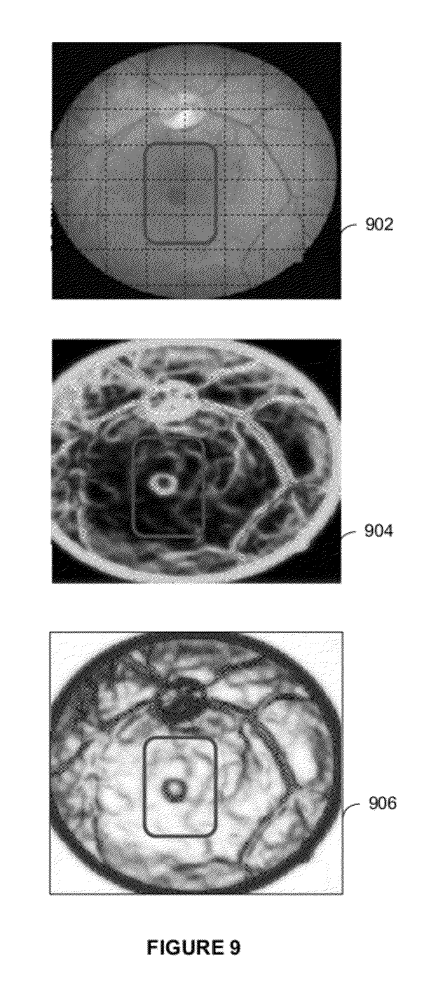

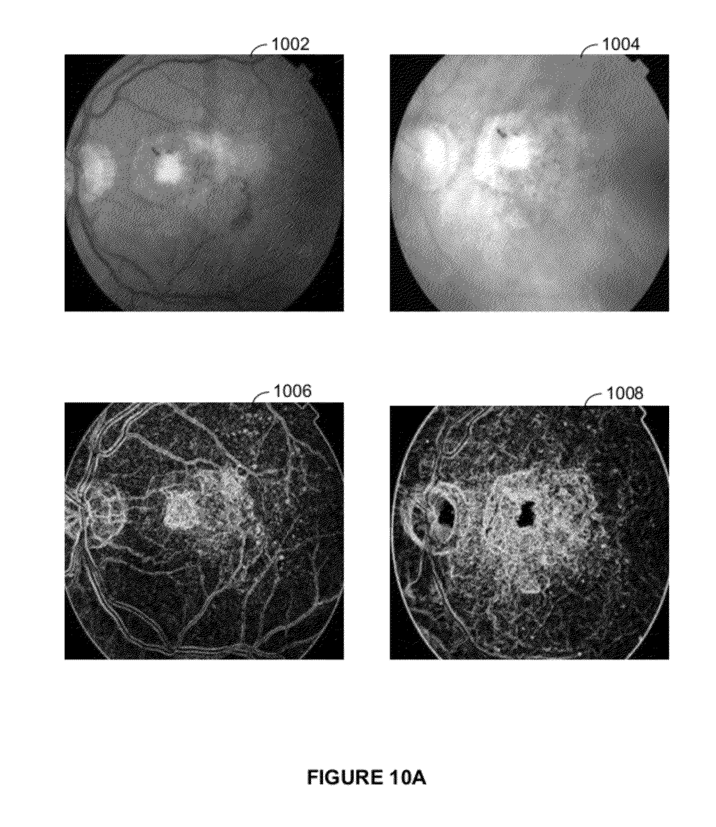

An example of a central idiopathic macular hole, a subretinal feature that is indicative of a TBI, is illustrated in FIG. 9. The top image 902 is a raw IR image of the retina, the middle image 904 is an attribute image generated from the raw IR image at the step 506 of FIG. 5, and the bottom image 906 is a color-reversed version of the attribute image 904. By employing the smoothing and segmenting techniques described herein, the system 300 is capable of quickly and clearly identifying features like macular holes, speeding up the diagnosis of TBI which may improve patient outcomes.

In some implementations, the process 500 includes additional image analysis or display steps that extract and provide information derived from multi-modal imaging (for example, extracting and analyzing information from visible light and IR images). For example, at the step 502, a plurality of images may be received, which may correspond to different or similar imaging modalities. In some implementations, the smoothing and segmenting framework discussed above with reference to the step 504 is configured for fusing and analyzing information extracted from multiple imaging modalities (or from multiple wavelengths of one imaging modality, such as multiple IR wavelengths). One way in which this framework is configured for multi-modal imaging application is by including additional terms in an energy functional formulation that address the smoothing and segmenting of images from the additional modalities. The additional terms may take the same form as the terms used for the first image or may take modified forms (e.g., using a different norm to evaluate distance, using different neighborhoods, or assigning different weights to different terms). Information from multiple imaging modalities may also be fused after smoothing and segmenting. In some implementations, the attribute images are computationally fused by summing, averaging, or by using any other combination technique. Images can be presented in two or three dimensions, and fused as topographic images with images obtained from optical coherent tomography, RGB images, and other IR images including those obtained by various scanning laser ophthalmoscopes, for example. In some implementations, the smoothing and segmenting framework discussed above with reference to the step 504 is configured for fusing and analyzing information extracted from an imaging modality (such as IR imaging) and information from a stored information source (such as information generated by other eye or physiological sensors, information from the scientific literature, demographic information, and previous images of the eye under study, for example).

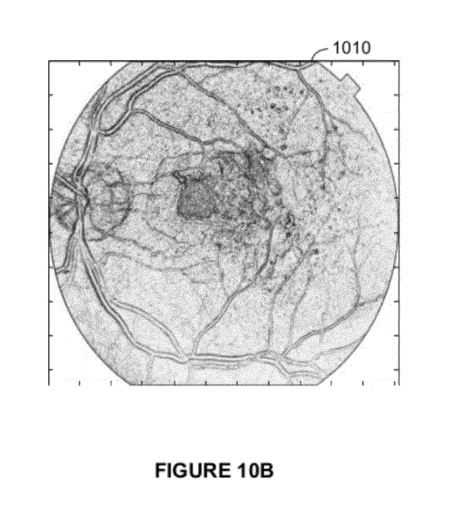

In some implementations, the attribute images are displayed concurrently for clinicians to visually analyze and compare. For example, FIG. 10A depicts visible and IR retinal images pre- and post-processing in accordance with the techniques described herein. Specifically, the visible light image 1002 (upper left) was used to generate an edge attribute image 1006 (lower left), and an IR light image 1004 (upper right) was used to generate an edge attribute image 1008 (lower right). The two sets of processed images provide different details that are useful to a clinician, and providing them side-by-side helps the clinician to identify the most relevant features of each. Here, the arteries are more distinctive in the processed visible light image 1006, while the processed IR light image 1008 provides clearer detail on the choroidal vasculature and the spread of subretinal or outer lesions. FIG. 10A illustrates the significant enhancements of various lesions associated with advanced AMD achievable with the systems and techniques disclosed herein. In particular, the area of chorioretinal scar (left central) is distinguished from surrounding areas associated with the dry form of AMD, in which the retinal tissue is still functional. In medical applications, the particular imaging modality used with the techniques and systems described herein may be chosen according to the patient's condition. In some implementations, the system 300 displays a combined image in which information from the first image of the eye is displayed with information from the second image of the eye, for example, in an overlay relationship. For example, FIG. 10B depicts a combined image 1010 in which information from multiple imaging modalities (here, different wavelengths of electromagnetic radiation) are displayed in an overlay relationship. Other modalities that may be used include flourescein angiography, indocyanine green angiography, OCT, enhanced-depth OCT, confocal scanning laser ophthalmoscopy, and autoflourescent devices.

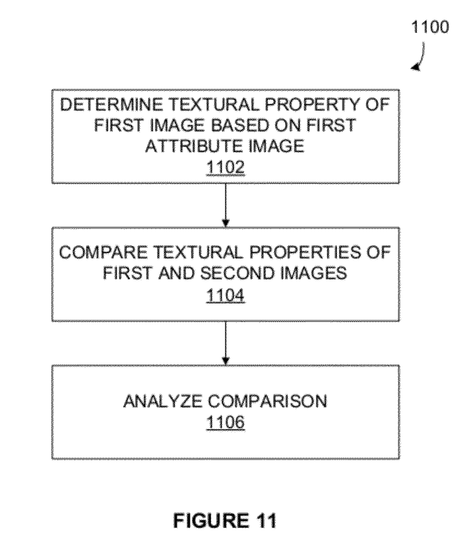

Furthermore, the systems and techniques disclosed herein can take into account additional medical information, imaging, laboratory tests, or other diagnostic information. In particular, the systems and techniques disclosed herein can detect changes across multiple images taken at different points in time. For example, in some implementations, the system 300 retrieves, from the image database 320, multiple images of the same subject taken at different times, and compare these images to detect changes in retinal or subretinal structure. FIG. 11 is a flow chart of a process 1100 for comparing first and second fundus images that may be executed by the automated decision support system 300 (FIG. 3). In preferred implementations, the system 300 executes the process 1100 after determining attributes of the first and second fundus images in accordance with the step 506 of FIG. 5, but other techniques for determining attributes may be used (for example, edge extraction, texture recognition, and pattern recognition).

At the step 1102, the system 300 determines a textural property of a portion of a first image of the eye based at least in part on the first attribute image. A textural property is a value of an attribute or a quantity derived from the value or values of one or more attributes. A textural property may be defined pixel-by-pixel in an image, or may be defined over a region within the first image (e.g., within the boundaries of an identified retinal or subretinal feature). In some implementations, the textural property is the value of the smoothed image generated by the system 300 at the step 504 of FIG. 5, the value of the edge field generated by the system 300 at the step 504 of FIG. 5, or a combination thereof. In some implementations, the textural property is represented by the coefficients of a wavelet decomposition of an attribute image. Any appropriate basis function and any number of terms in the wavelet decomposition may be used. Other image decomposition techniques, such as Fourier decomposition, may be used instead of or in addition to wavelet decomposition.

At the step 1104, the system 300 compares the first image of the eye to a second image of the eye by comparing the textural property of the portion of the first image of the eye (as determined at the step 1102) to a corresponding textural property of a corresponding portion of the second image of the eye. In some implementations, the system 300 compares the first and second images by performing a statistical change analysis, such as a t-statistic, to identify whether there is a statistically significant difference between the first and second images. The confidence intervals and other parameters for the statistical change analysis are selected according to the application. In implementations in which the first attribute image is decomposed into components at the step 1102, the system 300 compares the textural properties of the first and second images by comparing the respective decomposition coefficients to determine whether a statistically significant difference exists. Although the process 1100 is described with reference to multiple images of the same eye (e.g., taken at different times), the system 300 may be configured to implement the process 1100 to analyze images of different eyes of a same subject or images of eyes of different subjects, for example.

At the step 1106, the system 300 provides the result of the change analysis (e.g., to another support or analysis system, or to a clinician). In some implementations, the change analysis identifies one or more portions of the first and second images which are different between the images and these portions are identified graphically or textually at the step 1106. In some implementations, the change analysis determines whether a statistically significant difference exists, and notifies a clinician of the presence of a difference to prompt further investigation. This time-based analysis can identify subtle changes in retinal or subretinal structure which may reflect any of a number of conditions, such as inadequate tissue perfusion and the formation or growth of lesions. The system 300, when configured to execute the process 1100, may be included in any of a number of medical diagnostic systems, including a disease progression tracking system, a treatment efficacy evaluation system, and a blood diffusion tracking system.

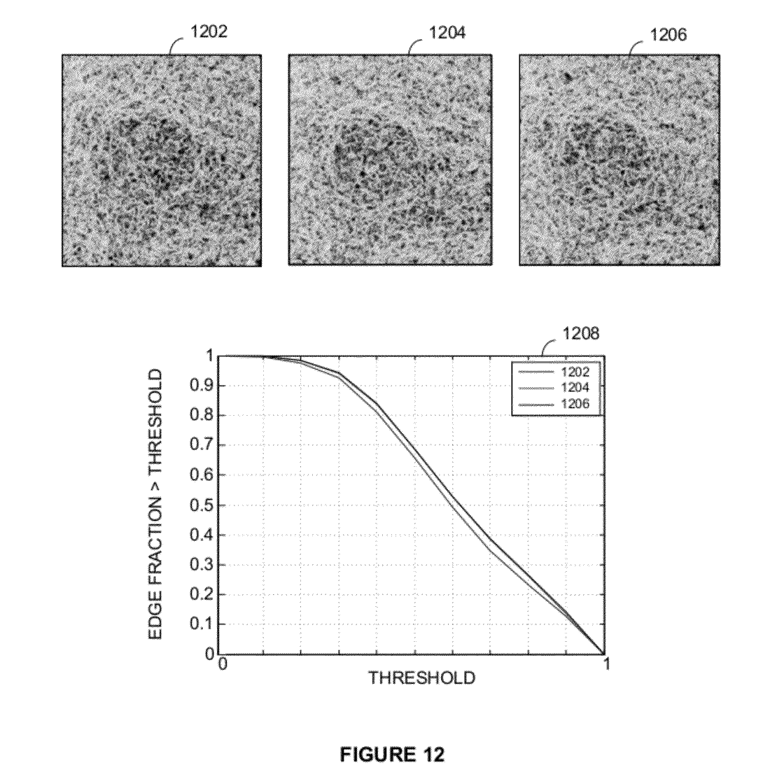

FIG. 12 depicts a first example of time-based image analysis according to process 1100 of FIG. 11. FIG. 12 depicts the 400×400 pixel edge fields of three IR fundus images 1202, 1204 and 1206, each of which have been smoothed and segmented in accordance with the step 504 of FIG. 5 as described above. The system 300 normalizes the edge field values for each edge field attribute image so that the maximum edge field value has value one. Next, for a threshold value T between zero and one, the system 300 determined the fraction of pixels in each edge field attribute image with edge field values greater than T. This threshold-fraction determination was repeated for a range of values of T, with the result (referred to as the “edge intensity distribution”) presented in the plot 1208. The traces corresponding to the edge intensity distributions for the images 1204 and 1206 are nearly indistinguishable, but both are visually distinct from the edge intensity distribution for the image 1202. One way of quantifying the difference between the 1202 edge intensity distribution and the 1204 and 1206 edge intensity distributions is by calculating the area under each distribution; here, the area under the 1302 edge intensity distribution is approximately 5.5% smaller than the area under either the 1204 distribution or the 1206 distribution. This time-based behavior indicates that a clinically-significant eye event occurred between the time at which the 1202 image was captured and the time at which the 1204 image was captured, with the eye stabilizing between the time at which the 1204 image was captured and the 1206 image was captured. A clinician can use these results (presented quantitatively or qualitatively) to more effectively question and examine the subject, improving diagnosis speed and sensitivity.

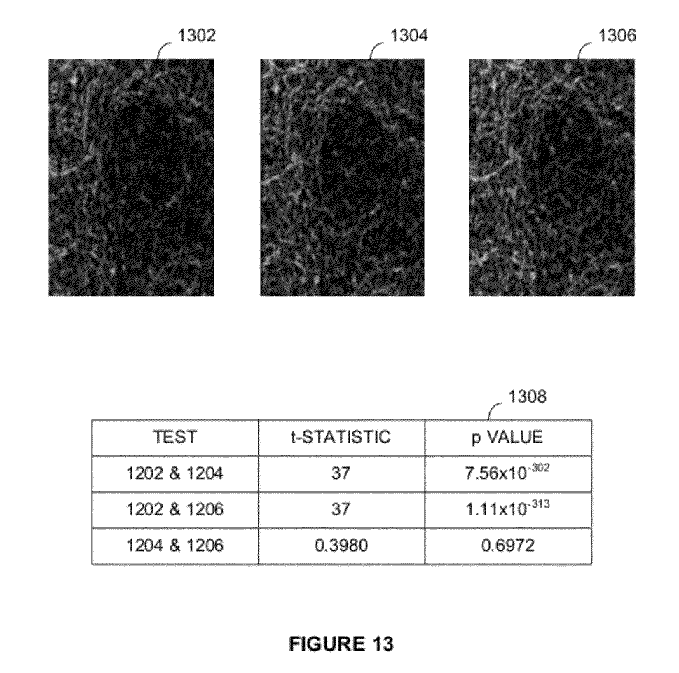

FIG. 13 depicts a second example of time-based image analysis according to process 1100 of FIG. 11. FIG. 13 depicts the edge fields of three sequentially-obtained IR fundus images 1302, 1304 and 1306, each of which have been smoothed and segmented in accordance with the step 504 of FIG. 5 as described above. The system 300 was configured to decompose each image using a wavelet decomposition and compare the wavelet coefficients using a t-statistic significance test, with the null hypothesis of no difference between the images. The results of the t-test are given in the table 1308 of FIG. 13. These results indicate a significant difference between the first image 1302 and the second image 1304, flagging a potentially clinically significant change in structure that is undetectable by the human eye.

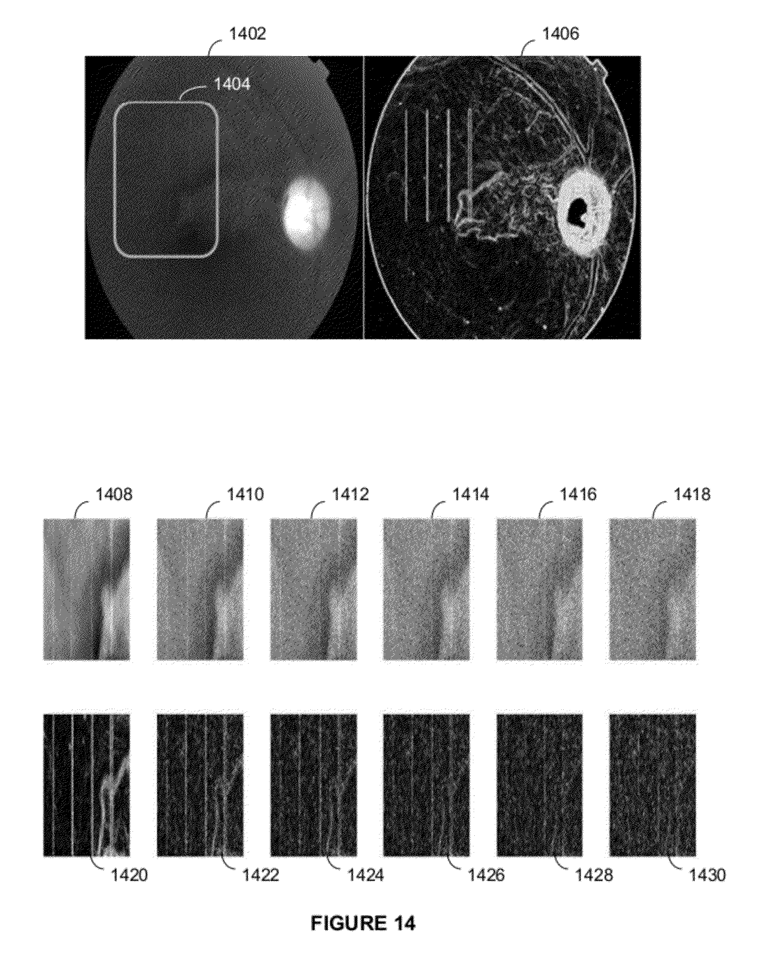

Compared to previous image analysis systems, the systems and techniques disclosed herein for image analysis exhibit an improved ability to detect fine features within images in the presence of noise. This detection capability is illustrated in FIG. 14 which depicts an IR fundus image 1402 of a subretinal scar to which four fictitious white lines (in the box 1404) were added. The white lines 1404 have widths of 1, 2, 4 and 10 pixels. To the human observer, these lines are hardly visible due to poor contrast in the image 1402. After undergoing smoothing and segmenting as described above with reference to step 504 of FIG. 5, the four lines are readily detected in the smoothed and segmented image 1406. The portions of the images 1402 and 1406 containing the added lines are reproduced in the small images 1408 and 1420. The five images 1410-1418 represent the image 1402 with increasing amounts of noise added. In particular, noise was added to the image 1402 at five different signal-to-noise ratios (SNRs): 2, 1, 0, −1 and −2 dB. Here, the SNR is defined in accordance with

SNR=10 log(I/σ) (4)

where I is the intensity of the white line added, and σ is the noise standard deviation. After undergoing smoothing and segmenting as described above with reference to step 504 of FIG. 5, the four lines are readily detected in the corresponding smoothed and segmented images 1422-1926, and still discernable in the images 1428 and 1430.
In some implementations, an IR image or a feature within an IR image, processed according to the process 500 of FIG. 5, is stored on a storage device as a library of features. The library of features may be used for feature identification, or for comparing features previously identified. In some implementations, the system 300 is configured to store a processed image using a sparse representation (for example, by executing a compressive sensing operation). Providing sparse representations of processed images allows the system 300 to readily compare the most significant characteristics of the images, aiding in quick and accurate comparisons.

### C. Information Extraction

As indicated in Sections A and B, the information extraction processor 330 of FIG. 3 is configured to extract information about the elements in an imaged scene by smoothing the image data to improve the representation of the scene, segmenting the image data to distinguish elements within the scene by determining edges between these elements, and estimating the attributes of the elements within the scene, using adaptively adjusted neighborhoods. This section describes a range of systems and methods for performing smoothing, segmenting and attribute determination as those operations are used in this disclosure. Additional techniques suitable for use with the system and methods disclosed herein are described by Desai and Mangoubi in U.S. Patent Application Publication No. 2009/0180693, incorporated by reference herein in its entirety.

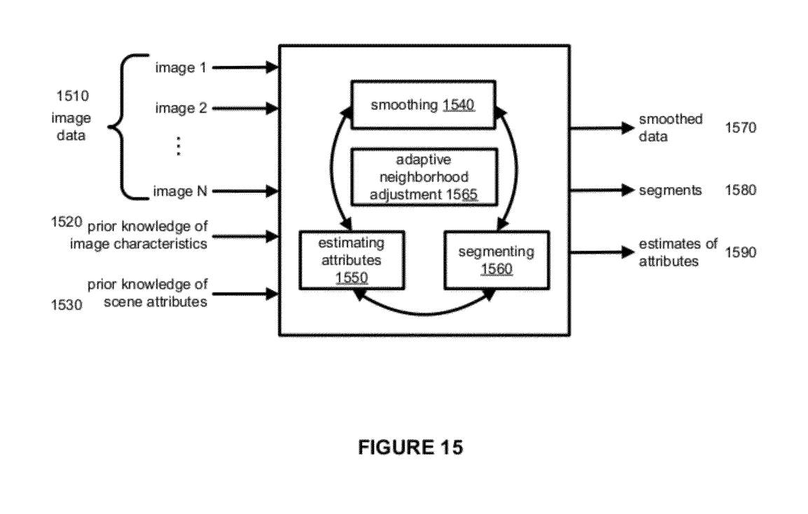

FIG. 15 depicts one illustrative implementation of the information extraction method performed by the information extraction processor 330. Inputs to the information extraction processor 330 include image data 1510 comprising images 1, 2, . . . , N; prior knowledge of the characteristics of the image data 1520; and prior knowledge of the attributes in the imaged scene 1530. Prior knowledge of the characteristics of the image data 1520 includes noise intensity and distribution information, models of the imaged scene, environmental factors, and properties of the imaging equipment. Prior knowledge of the attributes in the imaged scene 1530 includes locations within the scene that have known attributes, knowledge of the presence or absence of elements within the imaged scene, real-world experience with the imaged scene, or any probabilistic assessments about the content of the imaged scene. The processes of smoothing 1540, segmenting 1550 and attribute estimation 1560 are interdependent in the sense that the processor considers the outcome of each of these processes in performing the others. Adaptive adjustment of neighborhoods 1565 will be discussed in greater detail below. In addition, the processes are carried out concurrently or substantially concurrently. At the conclusion of these processes, the information extraction processor 330 outputs a collection of processed data comprising a set of smoothed data 1570, a set of segments dividing the imaged scene into coherent elements 1580, and a set of estimated attributes present within the scene 1590. Each of the processes 1540, 1550, and 1560 will be discussed in more detail below.

The smoothing process 1540 generates a set of smoothed data 1570 from the image data. Smoothed data 1570 represents the most accurate estimate of the true characteristics of the imaged scene. Images are often corrupted by noise and by distortions from the imaging equipment, and consequently, the image data is never a perfect representation of the true scene. When performing smoothing 1540, the processor 330 takes into account, among other factors, the image data, physical models of the imaged scene, characteristics of the noise arising at all points between the imaged scene and the database 320, as well as the results of the segmenting process 1550 and attribute estimation process 1560.

The segmenting process 1550 demarcates distinct elements within the imaged scene by drawing edges that distinguish one element from another. For example, in some implementations, the segmenting process distinguishes between an object and its background, several objects that overlap within the imaged scene, or regions within an imaged scene that exhibit different attributes. The segmenting process results in a set of edges that define the segments 1580. These edges may be scalar, vector, or matrix-valued, or may represent other data types. When performing segmenting 1550, the information extraction processor 330 takes into account, among other factors, the image data 1510, physical models of the imaged scene, characteristics of the noise arising at all points between the imaged scene and the image database 320, as well as the results of the smoothing process 1540 and attribute estimation process 1560.

The attribute estimation process 1560 identifies properties of the elements in the imaged scene. An attribute is any property of an object about which the image data contains some information. The set of available attributes depends upon the imaging modalities represented within the image data. For example, a thermographic camera generates images from infrared radiation; these images contain information about the temperature of objects in the imaged scene. Additional examples of attributes include texture, radioactivity, moisture content, color, and material composition, among many others. For example, the surface of a pineapple may be identified by the processor as having a texture (the attribute) that is rough (a value of the attribute). In one implementation, the attribute of interest is the parameter underlying a parameterized family of models that describe the image data. In another implementation, the attribute of interest is the parametric model itself. When performing attribute estimation, the information extraction processor 330 takes into account, among other factors, the image data 1510, physical models of the imaged scene, characteristics of the noise arising at all points between the imaged scene and the image database 320, as well as the results of the smoothing process 1540 and segmenting process 1550.

In some implementations, when more than one image is represented in the image data, the information extraction processor 330 determines, for a particular attribute, the relative amounts of information contained in each image. When estimating this attribute, the information extraction processor 330 utilizes each image according to its information content regarding the attribute. For example, multi-spectral imaging returns multiple images, each of which was produced by a camera operating in particular wavelength bands. Different attributes may be better represented in one frequency band than another. For example, satellites use the 450-520 nm wavelength range to image deep water, but the 1550-1750 nm wavelength range to image ground vegetation. Additionally, in some implementations, the information extraction processor 330 uses statistics of the image data to identify images of particular relevance to an attribute of interest. For example, one or more different weighted combinations of image data may be identified as having more information content as compared to other combinations for any particular attribute. The techniques disclosed herein allow the attribute estimation process, interdependently with the smoothing and segmenting processes, to preferentially utilize data from different images.

Additionally, in some implementations, the information extraction processor 330 preferentially utilizes data in different ways at different locations in the imaged scene for any of the smoothing, segmenting and attribute estimation processes. For example, if each image in a data set corresponds to a photograph of a person taken at a different angle, only a subset of those images will contain information about the person's facial features. Therefore, these images will be preferentially used by information extraction processor 330 to extract information about the facial region in the imaged scene. The information extraction method presented herein is capable of preferentially utilizing the image data to resolve elements in the imaged scene at different locations, interdependently with the smoothing, segmenting and attribute estimation processes.

It is important to note that the number of attributes of interest and the number of images available can be independent. For example, several attributes can be estimated within a single image, or multiple images may be combined to estimate a single attribute.

### D. Adaptive Neighborhooding

When producing a set of smoothed data 1570 from noisy images, or classifying segments according to their attribute values, it is desirable to be able to distinguish which locations within the imaged scene correspond to edges and which do not. When an edge is identified, the information extraction processor 330 can then treat locations on either side of that edge and on the edge itself separately, improving smoothing and classification performance. It is desirable, then, to use local information preferentially during the smoothing, segmenting and attribute estimation processes. Thus, in one implementation, decisions are made at each location based on a neighborhood of surrounding locations in an adaptive neighborhood adjustment process 1565. One implementation associates a neighborhood with each particular location in an imaged scene. Each neighborhood includes a number of other locations near the particular location. Information extraction processor 330 can then use the neighborhood of each location to focus the smoothing, segmenting and attribute estimation processes 1540-1560 to more appropriately extract information about the location. In its simplest form, the neighborhoods associated with each location could have a fixed size, shape and orientation, e.g. a circle with a fixed radius. However, using an inflexible neighborhood size and shape has a number of drawbacks. For example, if a location is located on an edge, then the smoothing and attribute estimation processes that rely on the fixed neighborhood will use information from the scene elements on either side of the edge, leading to spurious results. One improvement is adjusting the size of the neighborhood of each location based on local information. A further improvement comprises adjusting the size, shape and orientation of the neighborhood of a location to better match the local characteristics in an adaptive neighborhood adjustment process 1565. These examples will be described in greater detail below.

In one implementation, information extraction processor 330 performs the information extraction method while adjusting the size, shape and orientation characteristics of neighborhoods surrounding locations in the imaged scene. In particular, the processor 330 adapts the characteristics of the neighborhoods associated with each location interdependently with the smoothing, segmenting and attribute estimation processes 1540-1560. In another implementation, the information extraction processor 330 utilizes separate independently adapted neighborhoods for each attributed analyzed by the information extraction processor 330.

The benefits of using adaptive neighborhood size, shape and orientation can be seen in FIGS. 16A-16C. FIGS. 16A-16C illustrate three different neighborhood-based approaches. Each example of FIGS. 16A-16C depicts an edge and several illustrative neighborhoods 1610-1630 at corresponding locations. The first example illustrates an approach in which the neighborhoods 1610 associated with each location in the imaged scene are identical. In FIG. 16A, all neighborhoods 1610 are circles centered at the location with a fixed radius. In FIG. 16B, all neighborhoods 1620 are circular, but with radii that are allowed to vary in order to avoid a neighborhood 1620 overlapping an edge. In FIG. 16C, an exemplary implementation, neighborhoods 1630 are ellipses which are allowed to vary in their size, shape and orientation to better adapt to the characteristics of the local area, with the adaptation occurring interdependently with the smoothing process.

To demonstrate the improvement that such adaptation can provide, consider an exemplary implementation of the information extraction method which includes an averaging step within the smoothing process 1540 to reduce noise present in the raw image data. The averaging step produces a smoothed data value at each location (with an associated neighborhood) by replacing the image data value at that location with the average of the image data values at each of the locations that fall within the associated neighborhood.

With reference to FIGS. 16A-16C, this averaging will take place over the indicated neighborhoods 1610-1630. In FIG. 6A, averaging will occur over edge values and across segments, blurring the distinction between segments. A mathematical formulation in accordance with the neighborhood 410 is given by

$${min}_{u} {\int }_{R} ( α {u}_{X}^{T} {u}_{X} + {β ( u - g )}^{2} ) d X \qquad ( 5 )$$

wherein g is the image data, u is the smoothed data, α, β are adjustable parameters and the integral is taken over all locations X in region R.

In FIG. 16B, locations near the edge have associated neighborhoods 1620 that are necessarily small to avoid overlapping an edge, and thus are more susceptible to noise. A mathematical formulation in accordance with the neighborhood 1620 is given by

$${min}_{u , v} {\int }_{R} [ {α ( 1 - v )}^{2} {u}_{X}^{T} {u}_{X} + {β ( u - g )}^{2} + \frac{ρ}{2} {v}_{X}^{T} {v}_{X} + \frac{{v}^{2}}{2 ρ} ] d X \qquad ( 6 )$$

wherein g is the image data, u is the smoothed data, v is the edge values and α, β, ρ are adjustable parameters. A method related to that illustrated in FIG. 16B was used to analyze diffusion tensor imaging data of the human brain by Desai et al. in “Model-based variational smoothing and segmentation for diffusion tensor imaging in the brain,” Neuroinformatics, v. 4 2006, which is hereby incorporated by reference herein in its entirety.

In FIG. 16C, where size, shape and orientation are allowed to vary, averaging across an edge is prevented while allowing each location to selectively identify a neighborhood 1630 over which to average, improving noise-reduction performance. A mathematical formulation in accordance with the neighborhood 1630 is given by

$${min}_{u , V , w} {\int }_{R} [ α {{u}_{X}^{T} ( I - V )}^{2} {u}_{X} + {β ( 1 - w )}^{2} {‖ u - g ‖}_{2}^{2} + \frac{ρ}{2} F ( {V}_{X} ) + \frac{G ( V )}{2 ρ} + \frac{{ρ}_{w}}{2} {w}_{X}^{T} {w}_{X} + \frac{{w}^{2}}{2 {ρ}_{w}} ] d X \qquad ( 7 )$$

wherein g is the image data, u is the smoothed data; V is a symmetric, positive-definite 2×2 matrix representing the neighborhood; w weights the data fidelity terms; F and G are functions, and α, β, ρ, ρ_{w} are adjustable parameters. The information extraction processor 330 can also use information arising from the smoothing and attribute estimation processes 150-160 to adjust the size, shape and orientation of neighborhoods.

### F. Energy Functional Approach

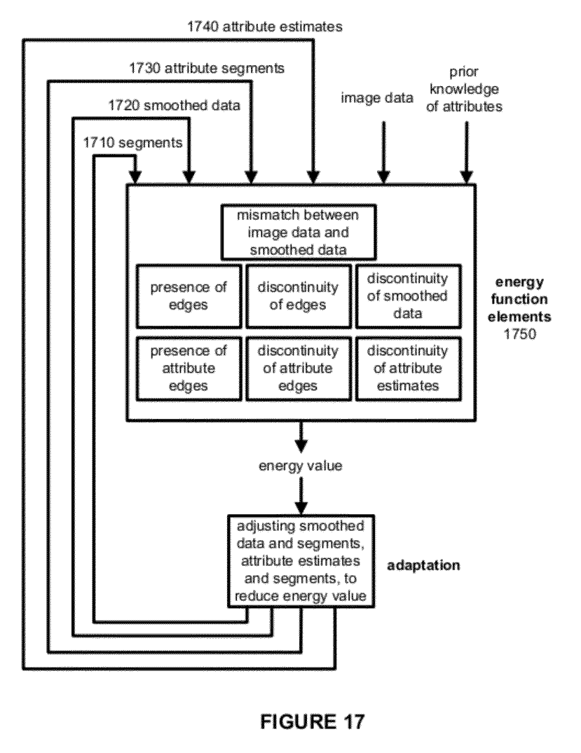

One particular implementation of the information extraction method is illustrated in FIG. 17. As discussed above, the neighborhood adaptation process can take place for each of the attributes of interest. At each location, a different neighborhood can be determined for each attribute, which allows the identification of attribute values and attribute edge values for each attribute. FIG. 17 depicts an iterative process which takes as inputs the image data, prior knowledge of attributes, segments (and associated edges) within the image data 1710, smoothed image data 1720, segments (and associated edges) within the attribute estimates 1730, and attribute estimates 1740. To begin to apply the iterative process of FIG. 17, initial values for the inputs 1710, 1720, 1730 and 1740 can be specified by a user or automatically selected by the processor 330. The adaptation process seeks to minimize an energy function which includes penalties for undesirable performance. Several example penalties that could be included in the energy function are depicted in energy function elements block 1750. These include penalties for mismatch between image data and smoothed data; penalties for the designation of excessive edges within the data; penalties for the designation of excessive edges within the attribute; penalties for the discontinuity or non-smoothness of edges within the data; penalties for the discontinuity or non-smoothness of edges within the attribute; discontinuity or abrupt changes in the smoothed data; and discontinuity or abrupt changes in attribute estimates. Using the inputs to the energy function, an energy value can be calculated, then inputs 1710, 1720, 1730 and 1740 are adaptively adjusted to achieve a lower energy value.

In one implementation of this implementation, the determination of the energy value is calculated in accordance with the following expression:

$${min}_{u , {υ}_{u} , θ , {υ}_{θ}} \int \int [ {e}_{1} + {e}_{2} + {e}_{3} + {e}_{4} + {e}_{5} ] \partial x \partial y \qquad ( 8 )$$

where e₁, e₂, e₃, e₄, e₅ are error terms as described below. Values for the smoothed data u, the edges of the segments νᵤ, attribute θ and the edges of the attribute segments ν_{θ}, are chosen for each (x, y) coordinate in order to minimize the expression contained in square brackets, integrated over the entire plane. This expression relies on the image data g, a data function T(θ) with attribute θ, and parameters λᵤ, αᵤ, ρᵤ, λ_{θ}, α_{θ}, ρ_{θ}, where

$${e}_{1} = {\vert g - T ( θ ) u \vert}^{2} , \\ {e}_{2} = {λ}_{u} {\vert ∇ u \vert}^{2} {( 1 - {υ}_{u} )}^{2} , \\ {e}_{3} = {α}_{u} ( {ρ}_{u} {\vert ∇ {υ}_{u} \vert}^{2} + \frac{{υ}_{u}^{2}}{{ρ}_{u}} ) , \\ {e}_{4} = {λ}_{θ} {\vert ∇ θ \vert}^{2} {( 1 - {υ}_{θ} )}^{2} , and$$

$${e}_{5} = {α}_{θ} ( {ρ}_{θ} {\vert ∇ {υ}_{θ} \vert}^{2} + \frac{{υ}_{θ}^{2}}{{ρ}_{θ}} ) .$$

The term e₁ is a penalty for a mismatch between the image data and the smoothed data, the term e₂ is a penalty for discontinuity in the smoothed data, the term e₃ includes penalties for the presence of an edge and the discontinuity of the edge, the term e₄ is a penalty for discontinuity in the attribute estimate and the term e₅ includes penalties for the presence of an attribute edge and the discontinuity of the attribute edge. One skilled in the art will recognize that there are many additional penalties that could be included in the energy function, and that the choice of appropriate penalties depends upon the application at hand. Equivalently, this problem could be expressed as the maximization of a reward function, in which different reward terms correspond to different desirable performance requirements for the information extraction method. There are many standard numerical techniques that could be readily applied to this specific mathematical formulation by one skilled in the art: for example, gradient descent methods. These techniques could be implemented in any of the implementations described herein.

In another implementation, the calculation of the minimum energy value is performed in accordance with the following expression:

$${min}_{u , w , {υ}_{m} , {υ}_{u} , {υ}_{c} , {θ}_{u} , {θ}_{m}} \int … \int \int [ {e}_{1} + {e}_{2} + {e}_{3} + {e}_{4} + {e}_{5} ] \partial {x}_{1} \partial {x}_{2} … \partial {x}_{N} \partial t \qquad ( 9 )$$

where e₁, e₂, e₃, e₄, e₅ are error terms as described below. Values for the smoothed data u, the edges of the segments w, the edge field of the measurement model parameters νₘ, the edge field of the process model parameters νᵤ, the edge field of the measurement model parameters νₘ, the edge field of the process parameter correlations νᵤ, the process model parameters θᵤ, and the measurement model parameters θₘ are chosen for each (x₁, x₂, . . . , x_{N}, t) coordinate in order to minimize the expression contained in square brackets, integrated over the entire N-dimensional image data space augmented with a one-dimensional time variable. The error terms are given by

e₁=βM(u,g,w,θₘ),

e₂=αₘLₘ(θₘ,νₘ)

e₃=αᵤCᵤ(u,νᵤ,θᵤ),

e₄=α_{c}L_{c}(ν_{c},θᵤ), and

e₅=π(u,w,νₘ,νᵤ,ν_{c},θᵤ,θₘ)

where M is a function that measures data fidelity, Lₘ estimates measurement model parameters, Cᵤ measures process model spatial correlation, L_{c} estimates process model parameters, π represents prior distributions of the unknown variables and β, αₘ, αᵤ, α_{c} are parameters that allow the process to place different emphasis on the terms e₁, e₂, e₃, e₄.

Additional image processing techniques may also be used with the smoothing, segmenting and attribute determination techniques described herein. For example, as discussed above, the image analysis techniques described herein can identify attributes of an image, such as texture. The Matrix Edge Onion Peel (MEOP) methodology may be used to identify features on the basis of their texture. In some embodiments, where textural regions are sufficiently large, a texture wavelet analysis algorithm may be used, but combined with an MEOP algorithm for textural regions of small size. This methodology is described in Desai et al., “Noise Adaptive Matrix Edge Field Analysis of Small Sized Heterogeneous Onion Layered Textures for Characterizing Human Embryonic Stem Cell Nuclei,” ISBI 2009, pp. 1386-1389, incorporated by reference in its entirety herein. An energy functional approach may be used for simultaneous smoothing and segmentation. The methodology includes two features: a matrix edge field, and adaptive weighting of the measurements relative to the smoothing process model. The matrix edge function adaptively and implicitly modulates the shape, size, and orientation of smoothing neighborhoods over different regions of the texture. It thus provides directional information on the texture that is not available in the more conventional scalar edge field based approaches. The adaptive measurement weighting varies the weighting between the measurements at each pixel.

In some embodiments, nonparametric methods for identifying retinal abnormalities may be used. These methods may be based on combining level set methods, multiresolution wavelet analysis, and non-parametric estimation of the density functions of the wavelet coefficients from the decomposition. Additionally, to deal with small size textures where the largest inscribed rectangular window may not contain a sufficient number of pixels for multiresolution analysis, the system 300 may be configured to perform adjustable windowing to enable the multiresolution analysis of elongated and irregularly shaped nuclei. In some exemplary embodiments, the adjustable windowing approach combined with non-parametric density models yields better classification for cases where parametric density modeling of wavelet coefficients may not applicable.

Such methods also allow for multiscale qualitative monitoring of images over time, at multiple spatiotemporal resolutions. Statistical multiresolution wavelet texture analysis has been shown to be effective when combined with a parametric statistical model, the generalized Gaussian density (GGD), used to represent the wavelet coefficients in the detail subbands. Parametric statistical multiresolution wavelet analysis as previously implemented, however, has limitations: 1) it requires a user to manually select rectangular, texturally homogeneous regions of sufficient size to enable texture analysis, and 2) it assumes the distribution of coefficients is symmetric, unimodal, and unbiased, which may be untrue for some textures. As described above, in some applications, the Matrix Edge Onion Peel algorithm may be used for small size irregularly shaped structures that exhibit “onion layer” textural variation (i.e., texture characteristics that change as a function of the radius from the center of the structure).

In some embodiments, an algorithm may be used to automatically segment features, and an adjustable windowing method may be used in order to maximize the number of coefficients available from the multiresolution decomposition of a small, irregularly shaped (i.e. non rectangular) region. These steps enable the automatic analysis of images with multiple features, eliminating the need for a human to manually select windows in order to perform texture analysis. Finally, a non-parametric statistical analysis may be applied to cases where the parametric GGD model is inapplicable. This analysis may yield superior performance over the parametric model in cases where the latter is not applicable.

A number of additional image processing techniques are suitable for use in the imaging systems and methods disclosed herein, including wavelet-based texture models, adaptive windowing for coefficient extraction, PDF and textural dissimilarity estimation, and density models such as the generalized Gaussian and symmetric alpha-stable, and KLD estimators such as the Ahmad-Linand or the Loftsgaarden-Quesenberry.

In some embodiments, more than one of the techniques described herein may be used in combination, for example, in parallel, in series, or fused using nonlinear classifiers such as support vector machines or probabilistic methods. Using multiple techniques for each retinal or subretinal feature may improve accuracy without substantially compromising speed.

The invention may be embodied in other specific forms without departing form the spirit or essential characteristics thereof. The forgoing embodiments are therefore to be considered in all respects illustrative, rather than limiting of the invention.

What is claimed is:

1. A method of imaging an eye, the method comprising:
receiving a first image of an eye of a subject, the first image being an infrared image or near-infrared image;
using a processing device, smoothing and segmenting the first image, wherein the smoothing and segmenting are interdependent;
determining a value of an attribute at a plurality of locations within the smoothed, segmented first image, the attribute indicative of at least one feature in the first image, the at least one feature including at least one of a retinal feature and a subretinal feature;
using a processing device, generating a first attribute image based at least in part on the determined values of the attribute; and
providing the first attribute image.

2. The method of claim 1, wherein segmenting the first image comprises identifying edge details within the first image.

3. The method of claim 1, wherein the first image comprises first and second images.

4. The method of claim 1, further comprising receiving a second image of the eye, the second image generated using a different imaging modality than used to generate the first image of the eye.

5. The method of claim 4, wherein the imaging modality used to generate the second image of the eye is visible light imaging.

6. The method of claim 4, wherein the smoothing, segmenting and determining comprises combining information from the first image of the eye with information from the second image of the eye.

7. The method of claim 6, further comprising displaying information from the first image of the eye with information from the second image of the eye.

8. The method of claim 1, wherein the smoothing, segmenting and determining comprises combining information from the first image of the eye with information from a stored information source.

9. The method of claim 1, wherein the first attribute image is provided to a display device and further comprising:
after providing the attribute image to the display device, receiving a triage category for the subject from a clinician.

10. The method of claim 1, wherein identifying the at least one feature is based at least in part on the first attribute image.

11. The method of claim 10, wherein identifying the at least one feature comprises identifying a boundary of the at least one feature based at least in part on the first attribute image.

12. The method of claim 10, wherein the at least one feature comprises a lesion, and further comprising providing quantitative information about the lesion.

13. The method of claim 1, wherein the at least one feature includes a zone 3 injury.

14. The method of claim 13, wherein the zone 3 injury includes at least one of a choroidal rupture, a macular hole, and a retinal detachment.

15. The method of claim 1, wherein the at least one feature is indicative of a traumatic brain injury.

16. The method of claim 1, wherein the at least one feature is indicative of at least one of age-related macular degeneration, retinal degeneration, retinal pigment epithelium degeneration, toxic maculopathy, glaucoma, a retinal pathology and a macular pathology.

17. The method of claim 1, further comprising:
determining a textural property of a portion of the first image of the eye based at least in part on the first attribute image; and
comparing the first image of the eye to a second image of a second eye by comparing the determined textural property of the portion of the first image of the eye to a textural property of a corresponding portion of the second image of the second eye.

18. The method of claim 17, wherein the first image and the second image represent one of: a same eye, different eyes of a same subject, and eyes of different subjects.

19. The method of claim 17, wherein the first image and the second image represent a same eye at two different points in time.

20. The method of claim 19, wherein the first attribute image is provided by at least one of a disease progression tracking system, a treatment efficacy evaluation system, and a blood diffusion tracking system.

21. The method of claim 17, wherein the textural properties of the respective portions of the first and second images of the eye are represented by coefficients of a wavelet decomposition, and comparing the first image of the eye to the second image of the eye comprises comparing the respective coefficients for a statistically significant difference.

22. The method of claim 17, wherein the textural properties of the respective portions of the first and second images are represented by respective first and second edge intensity distributions, and comparing the first image of the eye to the second image of the eye comprises comparing at least one statistic of the first and second edge intensity distributions.

23. The method of claim 1, wherein the segmenting and smoothing comprises determining an edge field strength at a plurality of locations in the image, and the attribute is based on the edge field strength.

24. The method of claim 23, wherein the edge field strength is based on a matrix edge field.

25. The method of claim 1, wherein providing the first attribute image comprises providing a sparse representation.

26. The method of claim 25, wherein providing a sparse representation comprises performing a compressive sensing operation.

27. The method of claim 25, further comprising:
storing, on a storage device, a plurality of features, each feature of the plurality of features represented by a sparse representation; and
comparing the identified at least one feature to the stored plurality of features.

28. A system for imaging an eye, comprising:
a processor configured to:
receive an electronic signal representative of a first image of an eye of a subject, the first image being an infrared image or near-infrared image;
smooth and segment the first image, wherein the smoothing and segmenting are interdependent;
determine a value of an attribute at a plurality of locations within the smoothed, segmented first image, the attribute indicative of at least one feature in the first image, the at least one feature including at least one of a retinal feature and a subretinal feature;
generate a first attribute image based at least in part on the determined values of the attribute; and
provide an electronic representation of the first attribute image.

29. The system of claim 28, wherein segmenting the first image comprises identifying edge details within the first image.

30. The system of claim 28, wherein the processor is further configured to receive an electronic signal representative of a second image of the eye, the second image generated using a different imaging modality than used to generate the first image of the eye.

31. The system of claim 30, wherein the imaging modality used to generate the second image of the eye is visible light imaging.

32. The system of claim 30, wherein the smoothing, segmenting and determining comprises combining information from the first image of the eye with information from the second image of the eye.

33. The system of claim 28, wherein identifying the at least one feature is based at least in part on the first attribute image.

34. The system of claim 33, wherein identifying the at least one feature comprises identifying a boundary of the at least one feature based at least in part on the first attribute image, and wherein the at least one feature comprises a lesion, the processor further configured to provide quantitative information about the lesion.

35. The system of claim 28, wherein the at least one feature is indicative of a traumatic brain injury.

36. The system of claim 28, wherein the at least one feature is indicative of at least one of age-related macular degeneration, retinal degeneration, retinal pigment epithelium degeneration, toxic maculopathy, glaucoma, a retinal pathology and a macular pathology.

37. The system of claim 28, wherein the processor is further configured to:
determine a textural property of a portion of the first image of the eye based at least in part on the first attribute image; and
compare the first image of the eye to a second image of a second eye by comparing the determined textural property of the portion of the first image of the eye to a textural property of a corresponding portion of the second image of the second eye.

38. The system of claim 37, wherein the processor is included in at least one of a disease progression tracking system, a treatment efficacy evaluation system, and a blood diffusion tracking system.

39. The system of claim 37, wherein the textural properties of the respective portions of the first and second images of the eye are represented by coefficients of a wavelet decomposition, and comparing the first image of the eye to the second image of the eye comprises comparing the respective coefficients for a statistically significant difference.

40. The system of claim 37, wherein the textural properties of the respective portions of the first and second images are represented by respective first and second edge intensity distributions, and comparing the first image of the eye to the second image of the eye comprises comparing at least one statistic of the first and second edge intensity distributions.

41. The system of claim 28, further comprising:
storing, on a storage device, a plurality of features, each feature of the plurality of features represented by a sparse representation; and
comparing the identified at least one feature to the stored plurality of features.

42. Non-transitory computer readable media storing computer executable instructions, which when executed by such a computer, cause the computer to carry out a method comprising:
receiving a first image of an eye of a subject, the first image being an infrared image or near-infrared image;
using a processing device, smoothing and segmenting the first image, wherein the smoothing and segmenting are interdependent;
determining a value of an attribute at a plurality of locations within the smoothed, segmented first image, the attribute indicative of at least one feature in the first image, the at least one feature including at least one of a retinal feature and a subretinal feature;
using a processing device, generating a first attribute image based at least in part on the determined values of the attribute; and
providing the first attribute image.
